(19) United States
(12) Patent Application Publication
(10) Pub. No.: US 2019/0269626 A1
(43) Pub. Date: Sep. 5, 2019

(54) Hair Protection Effect of Vanillyl Alkyl Ether and the Application thereof

(71) Applicant: CORUM INC., New Taipei City (TW)

(72) Inventors: Pei-Hsuan Wu, New Taipei City (TW); Tzu-Shuan Liu, New Taipei City (TW); Ssu-Ching Wang, New Taipei City (TW)

(21) Appl. No.: 16/286,693

(22) Filed: Feb. 27, 2019

Related U.S. Application Data
(60) Provisional application No. 62/638,386, filed on Mar. 5, 2018.
(60) Provisional application No. 62/637,430, filed on Mar. 2, 2018.

(51) Int. Cl. A61K 31/085; A61P 17/14

(52) U.S. Cl. CPC A61K 31/085; A61P 17/14

(57) ABSTRACT

This invention discloses a hair protection effect of vanillyl alkyl ether and the application thereof. The mentioned application comprises the process of topically applying a composition comprising vanillyl alkyl ether compound to reduce hair loss.

### BACKGROUND OF THE INVENTION

### 1. Field of the Invention

The present invention is generally related to a hair protection composition, and more particularly to hair protection effect of vanillyl alkyl ether and the application thereof.

### 2. Description of the Prior Art

Hair loss is a general phenomenon for men and women. There are many ways causing hair loss, and there are many studies about how to therapy hair loss. “Hair Follicle” is a mini-organ of the skin which contains dermal papilla cells (DPC) in the bottom site surrounded with matrix cells (keratinocytes) and melanocytes. DPC play an important role to regulate matrix cell proliferation, differentiation and apoptosis during hair cycles, and matrix cells are responsible for forming the major structures of the hair shaft. So that, in order to reduce hair loss, reducing hair loss factors on hair follicle from intrinsic and extrinsic factors are both important targets.

Hair loss, also called alopecia, occurs from genetic to environmental factors such as heredity, hormone imbalance, infection and stress. Under these adverse circumstances, inflammatory cytokines, like interleukin-6 (IL-6), interleukin-8 (IL-8), granulocyte-macrophage colony-stimulating factor (GM-CSF) and tumor necrosis factor-α (TNF-α) will be increase to interrupt hair growth. Besides, overexpression of prostaglandin-endoperoxide synthesis 2 (PTGS2, also called COX-2) also leads to hair loss. The most common type of hair loss is androgenetic-alopecia (AGA) which results from androgen receptor (AR) activation. When AR is activated by dihydrotestosterone (DHT), transforming growth factor beta-1 (TGF-β1) and beta-2 (TGF-β2) will be up-regulated to terminate hair growth cycle. The 5-alpha reductase is the enzyme which converts testosterone into Dihydrotestosterone (DHT). Both testosterone and DHT can bind to androgen receptor (AR), but DHT has much higher affinity to AR compared to testosterone. The expression of 5-alpha reductase is found in keratinocytes and fibroblasts in sebaceous and apocrine glands. It is also presented in hair follicles and in the prostate.

To one skilled in this art, abnormal DHT concentration will stimulate excessive sebum production in skin pore, allow bacterial growth and further lead to inflammation, infection and visible acne. Moreover, DHT is the main cause of hair loss in both men and women through up-regulating of IL-6 secretion, decreasing matrix cells proliferation, reducing the size of hair follicle and shortening the hair growth cycle. Therefore, the modulation of 5-alpha reductase activity is the main point to improve hair loss and skin acne formation.

Besides, Air pollutants-induced toxic effects occur mainly through Aryl hydrocarbon receptor (AhR) activation coming with reactive oxygen species (ROS) accumulation and inflammatory cytokine secretion. AhR is a ligand-activated transcription factor which serves as a biological sensor for many toxic chemical compounds, for example, polycyclic aromatic hydrocarbons (PAHs), polychlorinated biphenyl (PCB), and heavy metals. Cytochrome P450 1A1 (CYP1A1) and 1B1 (CYP1B1) are downstream AhR target genes involved in detoxification of toxic chemical compounds.

In view of the above matters, developing a novel composition of hair protection having the advantage of efficiently inhibiting hair loss and increasing hair growth factors is still an important task for the industry.

### SUMMARY OF THE INVENTION

In light of the above background, in order to fulfill the requirements of the industry, the present invention provides a novel hair protection effect of vanillyl alkyl ether and the application thereof having the advantage of efficiently improving hair loss. Moreover, the mentioned vanillyl alkyl ether also can prevent skin acne formation.

One objective of the present invention is to provide a hair protection effect of vanillyl alkyl ether and the application thereof to improve hair loss through inhibiting IL-6 and IL-8.

Another objective of the present invention is to provide a hair protection effect of vanillyl alkyl ether and the application thereof to improve hair loss through inhibiting pollutant caused risk on HFDPC.

Still another objective of the present invention is to provide a hair protection effect of vanillyl alkyl ether and the application thereof to improve hair loss through decreasing generation of reactive oxygen species (ROS).

Still another objective of the present invention is to provide a hair protection effect of vanillyl alkyl ether and the application thereof to improve hair loss and skin acne formation through inhibiting 5-alpha reductase activity with an economic way.

Still another objective of the present invention is to provide a hair protection effect of vanillyl alkyl ether and the application thereof to provide a new function of VBE on inhibiting 5-alpha reductase activity.

Accordingly, the present invention discloses a hair protection effect of vanillyl alkyl ether and the application thereof. The mentioned application comprises the process of topically applying a composition comprising vanillyl alkyl ether compound, the general structure of the mentioned vanillyl alkyl ether compound is as following:

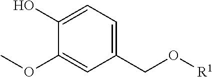

wherein R¹ represents a C₁ to C₈ alkyl group.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1A to FIG. 1B shows diagrams of reducing LPS-induced inflammatory cytokines (IL-6 and IL-8) in macrophages by VBE, Capsaicin, and Nonivamide of this invention;

FIG. 2A to FIG. 2B shows diagrams of reducing PMA-induced inflammatory cytokines (IL-6 and IL-8) in HFDPC (human follicle dermal papilla cells) by VBE while Capsaicin and Nonivamide have no effect of this invention;

FIG. 2C to FIG. 2D shows diagrams of reducing PMA-induced inflammatory cytokines (IL-6 and IL-8) in HFDPC by VBE while Minoxidil has no effect of this invention;

FIG. 3 shows diagram of reducing pollutant-induced hair loss related genes expression (CYP1B1, TGF-β2, and TNF-α) in HFDPC by VBE, and DMF of this invention;

FIG. 4A to FIG. 4B shows diagrams of reducing with/without pollutant-triggered ROS generation in HFDPC by VBE while CAP (capsaicin) has no effect of this invention;

FIG. 5 shows a diagram of how to improve hair loss of human hair follicle dermal papilla cells with VBE of this invention;

FIG. 6 shows a table presenting effect of compounds on DHT/Testosterone ratio in NHEK (Normal Human Epidermal Keratinocytes) of this invention;

FIG. 7 shows a diagram effect of compounds on DHT/Testosterone ratio in NHEK of this invention;

FIG. 8 shows a diagram of raw data of the example of FIG. 6 of this invention;

FIG. 9 shows table presenting 11 healthy subjects from 25 to 49 years with a chronic hair loss/alopecia of androgenetic origin for the following clinical evaluation of this invention;

FIG. 10 shows table presenting effect of clinical evaluation of the effect of VBE versus placebo on anagen hairs and telogen hairs in the subjects of FIG. 9 of this invention; and

FIG. 11 shows a diagram of clinical evaluation result of the effect of VBE versus placebo of FIG. 10 of this invention.

### DESCRIPTION OF THE PREFERRED EMBODIMENTS

What probed into the invention is hair protection effect of vanillyl alkyl ether and the application thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses an application of vanillyl alkyl ether on hair protection. The mentioned application comprises a composition comprising vanillyl alkyl ether compound for inhabiting hair loss, the general structure of the mentioned vanillyl alkyl ether compound is as following:

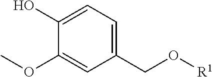

wherein R¹ is selected from C₁ to C₈ alkyl group.

In one preferred example of this embodiment, the mentioned vanillyl alkyl ether compound is vanillyl butyl ether (herein after presented as VBE).

Another preferred embodiment according to this specification discloses an application of vanillyl alkyl ether on hair protection. The mentioned application can be a method for inhibiting hair loss. The mentioned method for inhibiting hair loss comprises the process of topically applying a composition comprising vanillyl alkyl ether compound for inhabiting hair loss, the general structure of the mentioned vanillyl alkyl ether compound is as following:

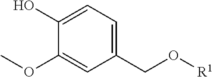

wherein R¹ represents a C₁ to C₈ alkyl group.

In one preferred example of this embodiment, the mentioned vanillyl alkyl ether compound is vanillyl butyl ether (herein after presented as VBE).

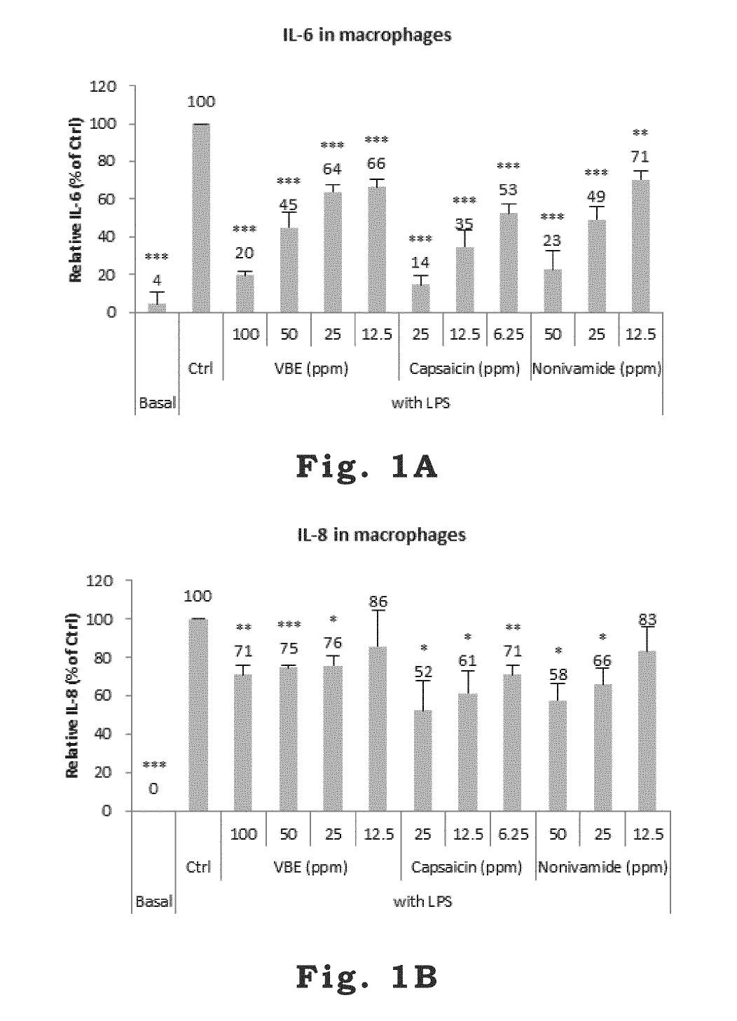

One preferred example of this embodiment is application of VBE on decreasing LPS-induced inflammation (IL-6, and IL-8). FIG. 1A to FIG. 1B respectively presents LPS-induced inflammation (IL-6, and IL-8) in macrophages individually decreased by VBE, Capsaicin, and Nonivamide. It can be found that VBE can decrease LPS-induced IL-6, and IL-8.

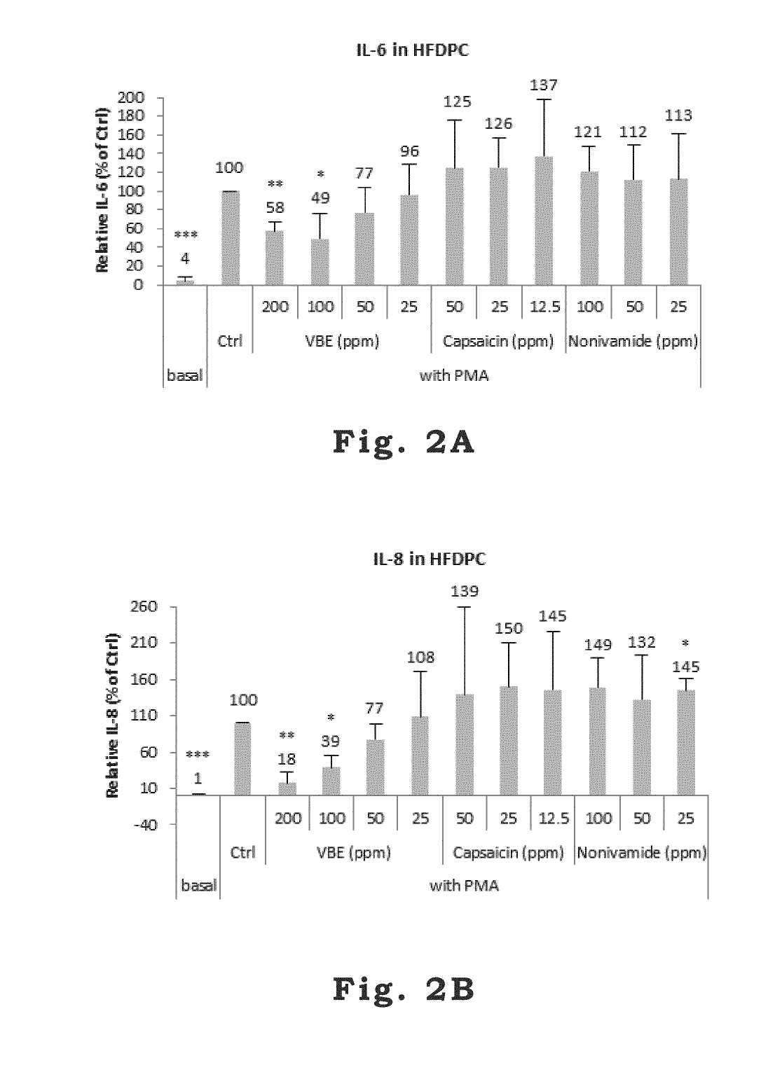

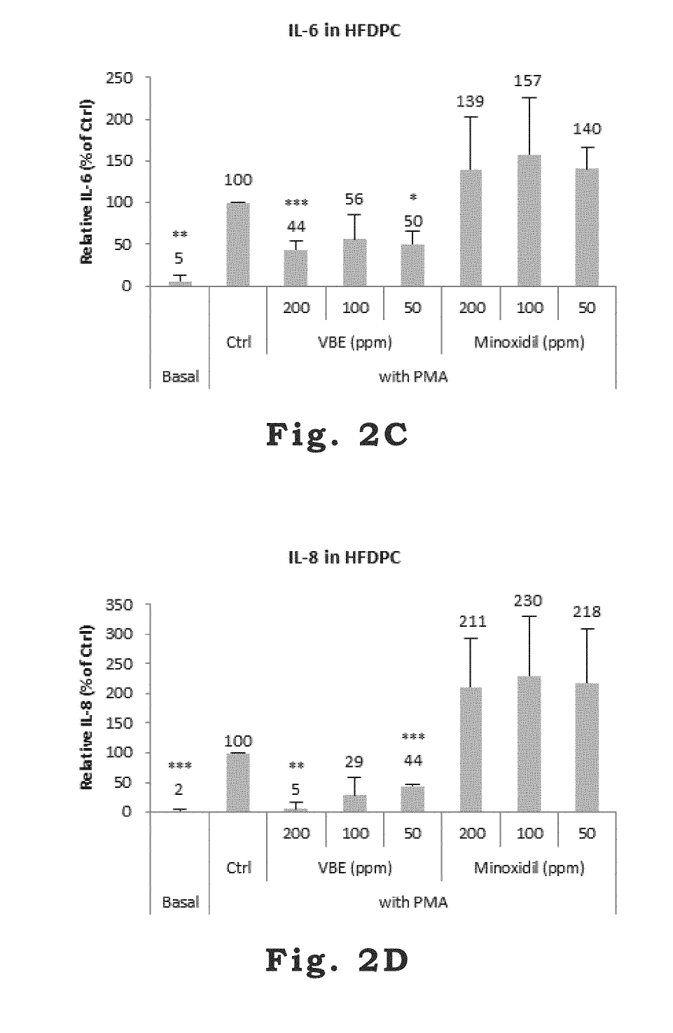

Still another example of this embodiment is application of VBE on decreasing PMA-induced inflammation (IL-6 and IL-8). FIG. 2A to FIG. 2B respectively presents PMA-induced inflammation (IL-6 and IL-8) in HFDPC individually decreased by VBE while Capsaicin and Nonivamide have no effect on PMA-induced inflammation. FIG. 2C to FIG. 2D respectively presents PMA-induced inflammation (IL-6, and IL-8) in HFDPC individually decreased by VBE, while Minoxidil has no effect on PMA-induced inflammation. From FIG. 2A to FIG. 2D, it can be found that VBE can decrease PMA-induced IL-6 and IL-8. Moreover, it also can be found that only VBE can reduce PMA-induced IL-6 and IL-8. Capsaicin, nonivamide, and minoxidil are effectless to PMA-induced IL-6, and IL-8.

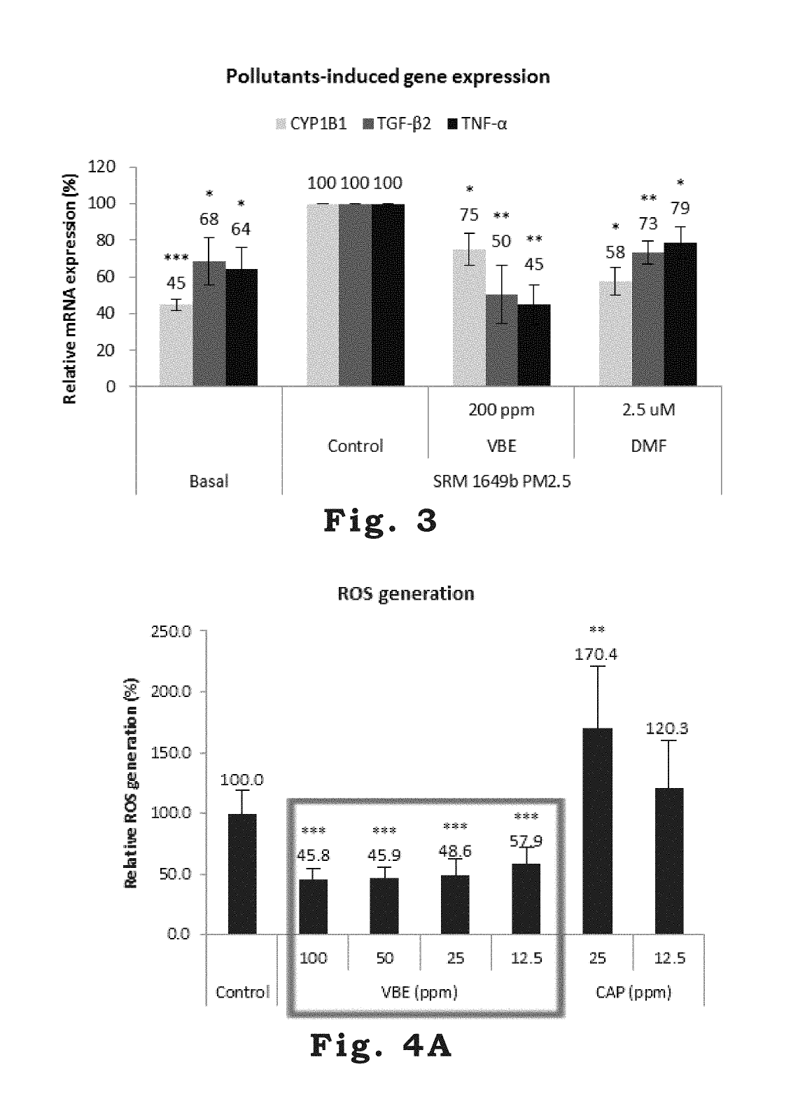

Still another example of this embodiment is application of VBE on decreasing pollutant-induced hair loss related genes expression (CYP1B1, TGF-β2, and TNF-α) on HFDPC. FIG. 3 presents pollutant-induced hair loss related genes expression (CYP1B1, TGF-β2, and TNF-α) by VBE, and DMF. DMF is 3′,4′-dimethoxyflavone, and is a AhR (aryl hydrocarbon receptor) antagonist. Pollutants usually generate damages though activating the transcription factor AhR. From FIG. 3, it can be found that VBE can reduce CYP1B1, TGF-β2, and TNF-α genes expression.

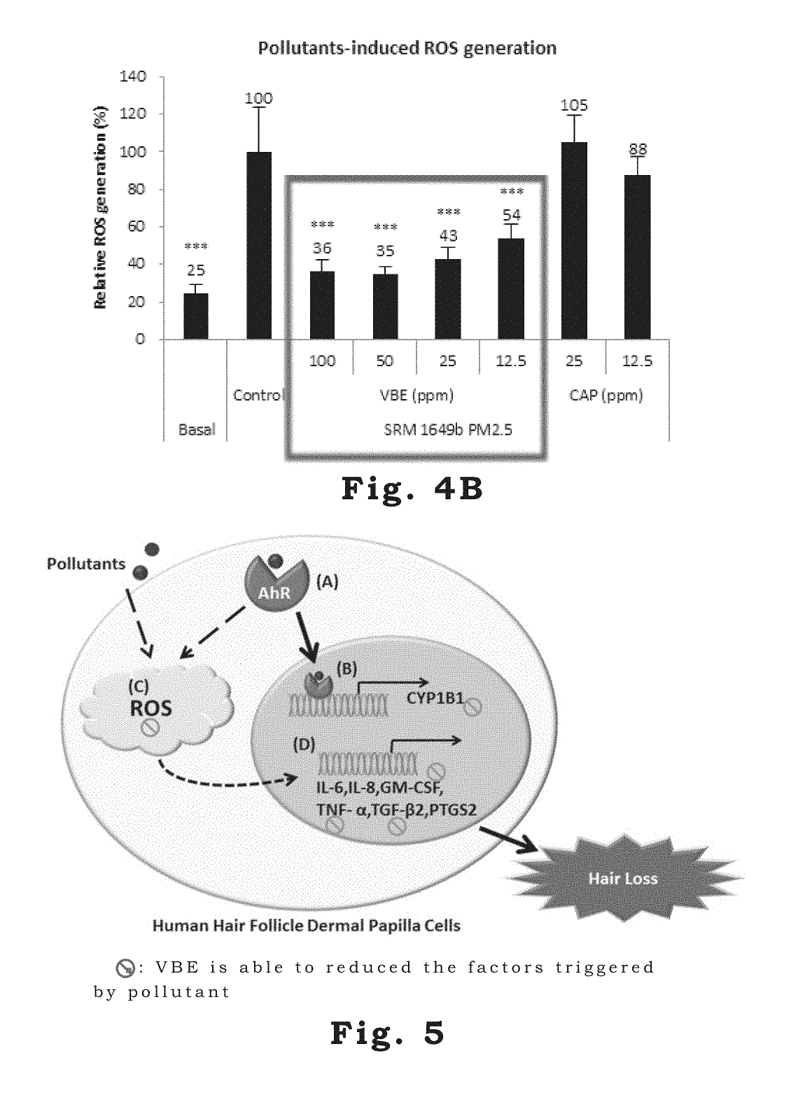

Still another example of this embodiment is application of VBE on decreasing with/or without pollutant-triggered ROS (reactive oxygen species) generation on HFDPC. FIG. 4A to FIG. 4B respectively presents with/without pollutant-triggered ROS generation on HFDPC by VBE, and CAP (capsaicin). From FIG. 4A to FIG. 4B, it can be found that only VBE decreased ROS in both basal and pollutant-stimulated condition.

FIG. 5 presents how to improve hair loss of human hair follicle dermal papilla cells with VBE according to the above examples. Hair follicle is a mini-organ of the skin which contains dermal papilla cells (DPC) in the bottom site surrounded with matrix cells (keratinocytes) and melanocytes. DPC play an important role to regulate matrix cell proliferation, differentiation and apoptosis during hair cycles, and matrix cells are responsible for forming the major structures of the hair shaft. In the above examples, we established an in vitro model to investigate hair loss mechanism caused by air pollution. Based on our data, SRM1649b (urban dust) PM2.5 is able to activate AhR pathway, enhance ROS generation, up-regulate gene expression of alopecia-related factors (TGF-β2, TNF-α and PTGS2) and stimulate inflammatory cytokines (IL-6, IL-8, and GM-CSF) in human hair follicle dermal papilla cells. In addition, VBE displayed the protective ability via attenuating AhR activity, ROS accumulation, GM-CSF production and mRNA levels of CYP1B1, TNF-α and TGF-β2. The mechanism of air pollution induced hair loss is illustrated in FIG. 5.

Still another preferred embodiment according to this specification discloses an application of vanillyl alkyl ether on hair protection through a method of inhibition of 5-alpha reductase activity. The mentioned method of inhibition of 5-alpha reductase activity comprises the process of topically applying a composition comprising vanillyl alkyl ether compound for inhibiting hair loss, the general structure of the mentioned vanillyl alkyl ether compound is as following:

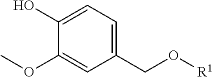

wherein R¹ represents a C₁ to C₈ alkyl group.

In one preferred example of this embodiment, the mentioned vanillyl alkyl ether compound is vanillyl butyl ether.

The following is an example, with general procedure of this embodiment, using vanillyl butyl ether on inhibiting 5-alpha reductase activity. In this example, Normal human epidermal keratinocytes (NHEK) are employed as the tested cells, and the Bioalternatives reference K341 is used at the 3^{rd} passage. The culture condition is under 37° C., and about 5% CO₂. The culture medium is Ketatinocyte-SFM supplemented with Epidermal growth factor (EGF) 0.25 ng/ml, Pituitary extract (PE) 25 μg/ml, and Gentamycin 25 μg/ml. The assay medium is Ketatinocyte-SFM supplemented with Gentamycin 25 μg/ml. According to this example, the experimental procedure is as following.

A. Culture and Treatment:

1. The cells were seeded in 24-well plate and grown in the culture medium for 24 hours.

2. The medium was replaced with the assay medium containing vanillyl butyl ether. In the controlled experiment, the medium is replaced with assay medium. In the reference experiment, the medium is replaced with the reference compound (Finasteride).

3. The Cells were pre-incubated for 24 hours.

4. After the pre-incubation time, the treatments were renewed, [¹⁴C] testosterone was added and cells were incubated for 24 hours further.

5. All experimental condition were performed in triplicate.

6. At the end of the incubation time, the supernatants were collected for testosterone metabolism analysis.

B. Extractions and Analysis:

1. The steroid molecules from supernatants were extracted with a chloroform/methanol mix.

2. The organic phase was collected and the different molecular species (testosterone metabolites) were separated by thin layer chromatography (TLC and using a solvent system containing dichloromethane, ethylacetate and methanol.

3. Autoradiography was performed on chromatography and the level of the transformed testosterone was estimated by densitometric analysis of the different spots corresponding to testosterone metabolites.

C. Data Management:

1. The inter-group comparisons were performed by an unpaired Student's t-test.

2. Standard error of the mean: sem=Sd/√n

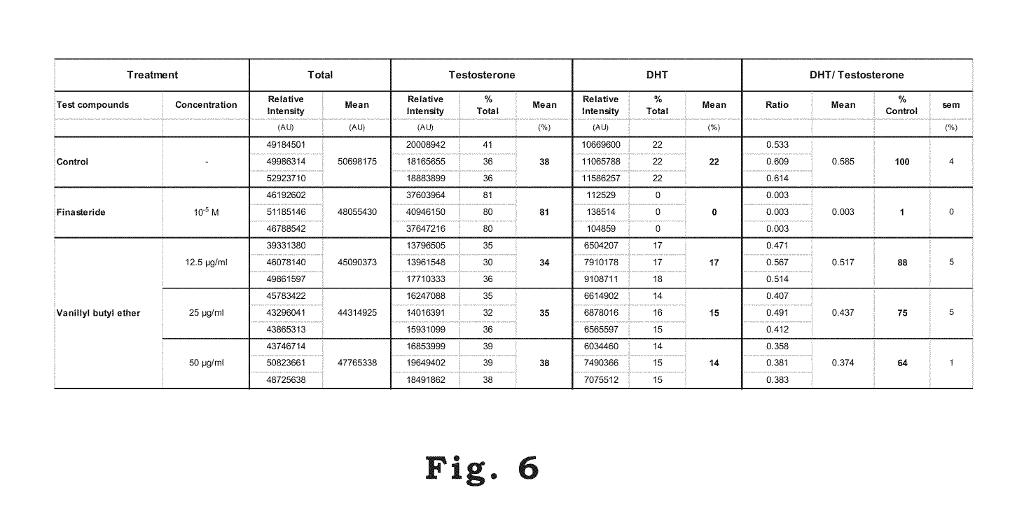

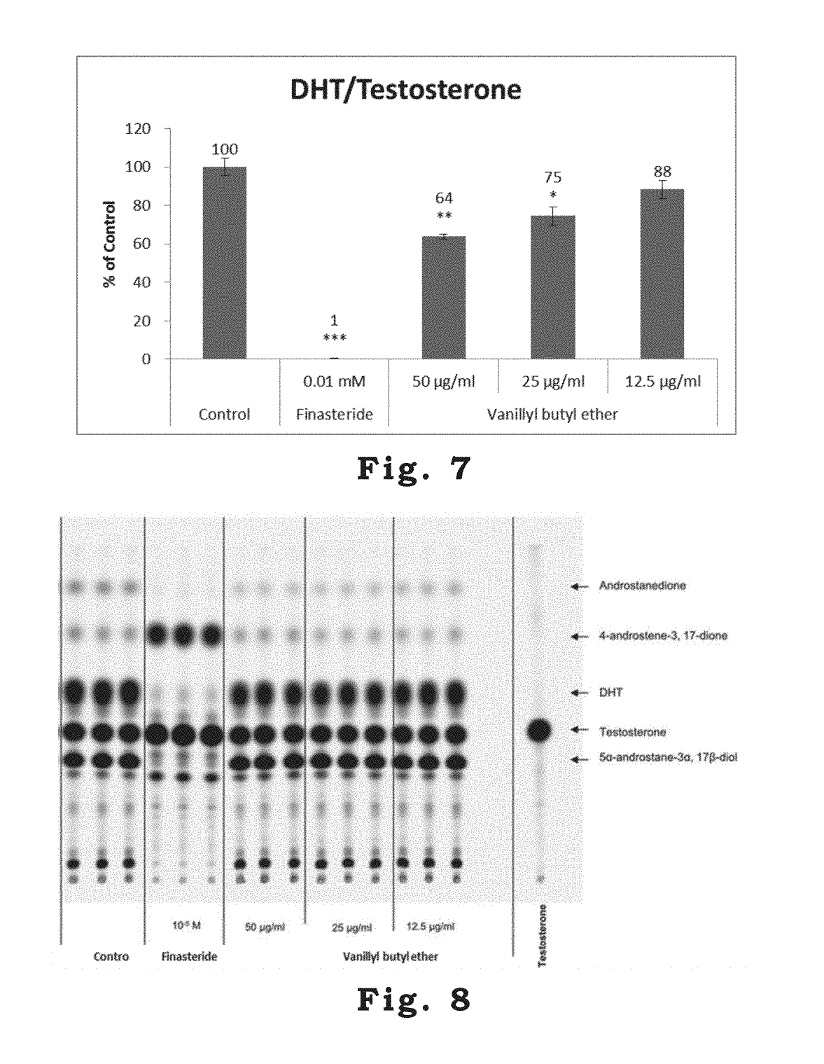

This test is assayed and reported by Bioalternatives. FIG. 6 shows a table presenting effect of compounds on DHT/Testosterone ratio in NHEK. FIG. 7 shows the effect of compounds on DHT/Testosterone ratio in NHEK. The raw data of this example is shown in FIG. 8.

In the presence of finasteride, the 5-alpha reductase inhibitor, the DHT/testosterone ratio represented only 1% compared to the control condition which was expected and validated the assay.

According to FIG. 7, 50 μg/ml vanillyl butyl ether reduces the DHT/Testosterone ratio to 64% compared to control. And the concentration of 1.25 to 50 μg/ml of vanillyl butyl ether decreases DHT/Testosterone ratio in a dose dependent manner which means vanillyl butyl ether suppresses 5-alpha reductase activity.

In conclusion, vanillyl butyl ether represented the inhibitory effect on 5-alpha reductase activity, demonstrating that it has potential to be used for anti-hair loss and anti-acne treatment.

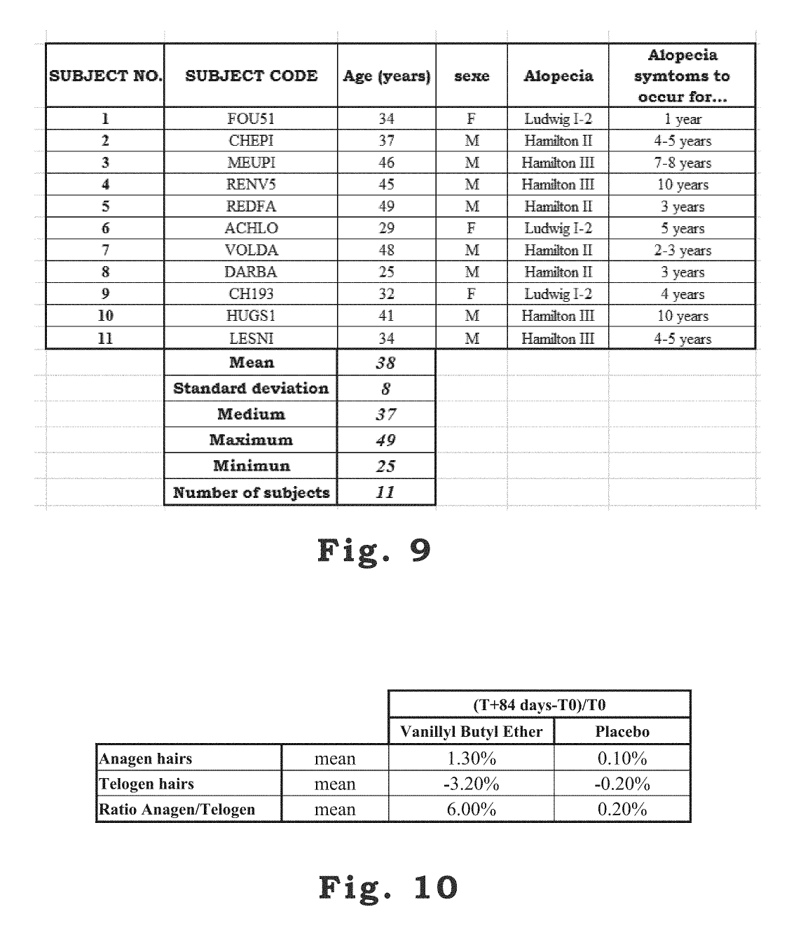

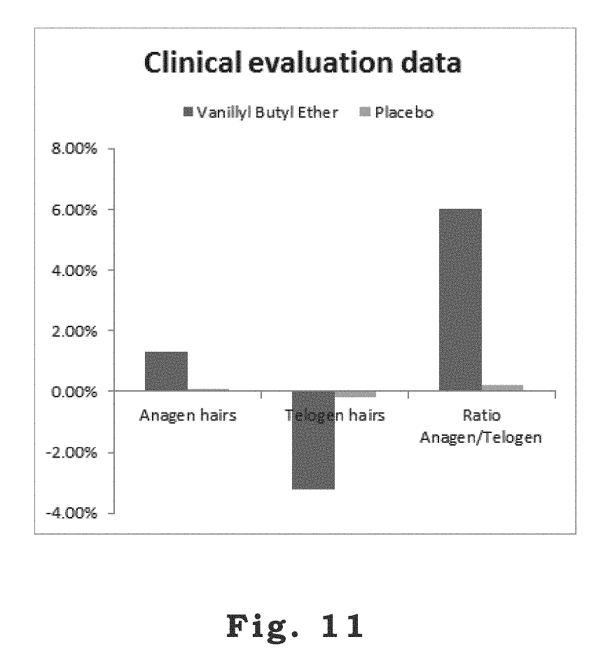

Another example of this embodiment is clinical evaluation of the effect of VBE versus placebo in the male and female subjects after repeated application of 84 days through. The clinical test is finished by Spincontrol, France. The evaluation was performed using phototrichogram for hair growth cycle analysis. 11 healthy subjects from 25 to 49 years with a chronic hair loss/alopecia of androgenetic origin were selected for this study. The details of subjects are presented in the table in FIG. 9. Vanillyl Butyl Ether or Placebo was applied once a day for 84 days on a randomized half-head (one product per half-head). The results showed that after 84 days of daily application, Vanillyl Butyl Ether increased Anagen (growing) hair, reduced Telogen (resting or shedding) hair and enhanced the ratio of Anagen to Telogen by 6% (as shown in FIG. 10 and FIG. 11).

In summary, we have reported a hair protection effect of vanillyl alkyl ether and the application thereof. The mentioned application comprises the process of topically applying a composition comprising vanillyl alkyl ether compound for inhibiting hair loss. According to this invention, the mentioned application can efficiently inhibit hair loss. Moreover, the mentioned application of this specification also can efficiently inhibit of 5-alpha reductase activity for not only improving hair loss, but also preventing skin acne formation.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

1. A method for inhibiting hair loss, comprising:
topically applying a composition to skin, wherein the composition comprising an effective amount of vanillyl alkyl ether having a structure represented in the following general formula: 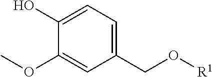
wherein R¹ represents a C₁ to C₈ alkyl group.

2. The method for inhibiting hair loss according to claim 1, wherein said vanillyl alkyl ether is vanillyl butyl ether.

3. The method for inhibiting hair loss according to claim 1, wherein said vanillyl alkyl ether is used on reducing PMA-induced inflammation IL-6, and IL-8.

4. The method for inhibiting hair loss according to claim 1, wherein said vanillyl alkyl ether is used on reducing ROS (reactive oxygen species) generation.

5. The method for inhibiting hair loss according to claim 1, wherein said vanillyl alkyl ether is used on reducing pollutant-induced ROS (reactive oxygen species) generation.

6. The method for inhibiting hair loss according to claim 1, wherein said vanillyl alkyl ether is used on reducing pollutant-induced hair loss related genes expression of CYP1B1, TGF-β2, and TNF-α.

7. The method for inhibiting hair loss according to claim 1, wherein said vanillyl alkyl ether is used on inhibiting 5-alpha reductase activity.

8. The method for inhibiting hair loss according to claim 1, wherein said vanillyl alkyl ether is used on decreasing DHT/Testosterone ratio.

9. A composition for inhibiting hair loss, comprising:
an effective amount of vanillyl alkyl ether having a structure represented in the following general formula: 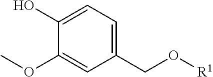
wherein R¹ represents a C₁ to C₈ alkyl group.

10. The composition for inhibiting hair loss growth according to claim 9, wherein said vanillyl alkyl ether is vanillyl butyl ether.

11. The composition for inhibiting hair loss according to claim 9, wherein said vanillyl alkyl ether is used on reducing PMA-induced inflammation IL-6, and IL-8.

12. The method for inhibiting hair loss according to claim 9, wherein said vanillyl alkyl ether is used on reducing ROS (reactive oxygen species) generation.

13. The method for inhibiting hair loss according to claim 9, wherein said vanillyl alkyl ether is used on reducing pollutant-induced ROS (reactive oxygen species) generation.

14. The composition for inhibiting hair loss according to claim 10, wherein said vanillyl alkyl ether is used on reducing pollutant-induced hair loss related genes expression of CYP1B1, TGF-β2, and TNF-α.

15. The composition for inhibiting hair loss according to claim 10, wherein said vanillyl alkyl ether is used on inhibiting 5-alpha reductase activity.

16. The composition for inhibiting hair loss according to claim 10, wherein said vanillyl alkyl ether is used on decreasing DHT/Testosterone ratio.
